(19) United States
(12) Patent Application Publication
(10) Pub. No.: US 2008/0160120 A1
(43) Pub. Date: Jul. 3, 2008

(54) LAPACTIC AGENT

(76) Inventors: TOSHIYA SAWAI, Yokohama-shi (JP); Kenji Arakawa, Yokohama-shi (JP); Masakazu Hatakeyama, Yokohama-shi (JP); Jun Hiraki, Tokyo (JP)

Correspondence Address: J.C. Patents, Suite 250, 4 Venture, Irvine, CA, 92618, US

(21) Appl. No.: 12/049,216

(22) Filed: Mar. 14, 2008

Related U.S. Application Data
(62) Division of application No. 10/464,859, filed on Jun. 16, 2003.

(30) Foreign Application Priority Data
Sep. 27, 2002 (JP) 2002-282936

(51) Int. Cl. A61K 36/00

(52) U.S. Cl. 424/773

(57) ABSTRACT

PROBLEMS TO BE SOLVED: To provide a highly safe lapactic agent that exhibits a sufficient lapactic effect such as the promotion of catharsis even by the intake of a small quantity, and a food product containing such a lapactic agent having a lapactic effect.

MEANS TO SOLVE THE PROBLEMS: A lapactic agent comprising (A) a basic polymer and/or the salt thereof, and (B) a dietary fiber, wherein (A) a basic polymer and/or the salt thereof is at least one selected from the group consisting of polyvinylamine, polyallylamine, polyethyleneimine, polyvinylpyridine, poly (N,N-dimethylaminoethyl acrylate), ε-polylysine, a copolymer formed from a monomer having an amino group selected from the group consisting of vinylamine, allylamine, vinylpyridine, vinylmethylimidazole, N,N-dimethylaminoethyl acrylate, and N,N-dimethylaminopropyl acrylate; and a salt of these polymers; and a food product containing such a lapactic agent.

### FIELD OF THE INVENTION

The present invention relates to a lapactic agent that is easily ingested and is effective in a small amount of intake; and to a food product that contains such a lapactic agent.

### DESCRIPTION OF THE PRIOR ART

Dietary fibers have been known to be difficult to be digested and absorbed in the gastrointestinal tract, to be swollen by absorbing mainly moisture in the intestines, and to have an effect of improving bowel movement, such as increasing the quantity of feces, accelerating peristaltic motions, or a cathartic effect. Although dietary fibers can be used safely, the effect thereof is too moderate; therefore, achieving the lapactic effect requires the intake of a large quantity of dietary fibers. Since dietary fibers, such as alginic acid, have a very low solubility to water, and gelate easily, they are difficult to ingest in a large quantity, and dietary fibers may worsen the feel of food products to the tongue and in the mouth. Excessive intake of dietary fibers may also have adverse effects such as the inhibition of systemic absorption. In order to solve these problems, the combined use of a plurality of dietary fibers, or dietary fibers and other substances has been proposed (see, for example, Patent References 1 and 2).

### [Patent Reference 1]

Japanese Patent Application Laid-Open No. 5-328938 (1993)

### [Patent Reference 2]

Japanese Patent Application Laid-Open No. 2000-281583

The object of the present invention is to provide a highly safe lapactic agent that exhibits a sufficient lapactic effect such as the promotion of catharsis even by intake in a small quantity, and a food product containing such a lapactic agent having a lapactic effect.

### SUMMARY OF THE INVENTION

The present inventors have conducted repeated studies, and as a result have found that a lapactic agent comprising (A) a basic polymer and/or the salt thereof, and (B) a dietary fiber, wherein (A) a basic polymer and/or the salt thereof is at least one selected from the group consisting of polyvinylamine, polyallylamine, polyethyleneimine, polyvinylpyridine, poly N,N-dimethylaminoethyl acrylate), ε-polylysine, a copolymer formed from a monomer having an amino group selected from the group consisting of vinylamine, allylamine, vinylpyridine, vinylmethylimidazole, N,N-dimethylaminoethyl acrylate, and N,N-dimethylaminopropyl acrylate; and a salt of these polymers, exhibits lapactic effects, such as increase in fecal quantities and the promotion of bowel movement even by a small quantity of intake, exhibits high safety, is easy to ingest, and is easily added to food products; and on the basis of these findings the present inventors have completed the present invention.

The present invention is constituted as follows:

(1) A lapactic agent comprising (A) a basic polymer and/or the salt thereof, and (B) a dietary fiber, wherein (A) a basic polymer and/or the salt thereof is at least one selected from the group consisting of polyvinylamine, polyallylamine, polyethyleneimine, polyvinylpyridine, poly (N,N-dimethylaminoethyl acrylate), ε-polylysine, a copolymer formed from a monomer having an amino group selected from the group consisting of vinylamine, allylamine, vinylpyridine, vinylmethylimidazole, N,N-dimethylaminoethyl acrylate, and N,N-dimethylaminopropyl acrylate; and a salt of these polymers.
(2) The lapactic agent according to (1), wherein (A) the basic polymer and/or the salt thereof is ε-polylysine and/or the salt thereof.
(3) The lapactic agent according to (1) or (2), wherein (B) the dietary fiber is at least one selected from the group consisting of apple fiber, sugar beet fiber, corn fiber, wheat fiber, and soybean fiber.
(4) A food product that contains a lapactic agent according to any of (1) to (3).

### DESCRIPTION OF THE PREFERRED EMBODIMENT

(A) the basic polymer and/or the salt thereof (hereafter referred to as (A) component) is at least one selected from the group consisting of polyvinylamine, polyallylamine, polyethyleneimine, polyvinylpyridine, poly (N,N-dimethylaminoethyl acrylate), ε-polylysine, a copolymer formed from a monomer having an amino group selected from the group consisting of vinylamine, allylamine, vinylpyridine, vinylmethylimidazole, N,N-dimethylaminoethyl acrylate, and N,N-dimethylaminopropyl acrylate; and an inorganic salt such as chloride and phosphate and an organic salt such as citrate and maleate of these polymers. The (A) component has characteristics of not being digested in the digestive tract or being difficult to digest in the digestive tract.

Of these polymers, ε-polylysine or the salt thereof is preferable as the (A) component, because when used together with a dietary fiber, it exhibits a significant synergistic effect for promoting bowel movement, and can be obtained from a natural material.

In the present invention, ε-polylysine used as the (A) component may be obtained by an arbitrary method. Specifically, ε-polylysine may be obtained by use of a method disclosed in U.S. Pat. No. 1,245,361 wherein Streptomyces Albulus Subspecies Lysinopolymerus is cultivated in a culture medium containing 5% by weight glucose, 0.5% by weight yeast extract, 1% by weight ammonium sulfate, 0.08% by weight dipotassium hydrogenphosphate, 0.136% by weight potassium dihydrogenphosphate, 0.05% by weight magnesium sulfate heptahydrate, 0.004% by weight zinc sulfate heptahydrate, and 0.03% by weight iron (II) sulfate heptahydrate; pH is adjusted to 6.8; and s-polylysine is isolated and collected from the obtained culture collection.

As the (A) component in the present invention, there may be used free ε-polylysine; an inorganic salt of e-polylysine formed from an inorganic acid, such as hydrochloric acid, sulfuric acid, or phosphoric acid, and ε-polylysine; an organic salt of ε-polylysine formed from an organic acid, such as acetic acid, propionic acid, fumaric acid, malic acid, citric acid, maleic acid, adipic acid, gluconic acid, or lactic acid, or ε-polylysine; a saturated fatty acid salt of ε-polylysine formed from a medium- or long-chain saturated fatty acid, such as capryonic acid, lauric acid or stearic acid, or ε-polylysine; an unsaturated fatty acid salt of ε-polylysine formed from a medium- or long-chain unsaturated fatty acid, such as oleic acid, linoleic acid, or arachidonic acid, or ε-polylysine; or a mixture thereof.

The toxicity of ε-polylysine and the salts thereof used in the present invention is very low; for example, the acute toxicity of ε-polylysine hydrochloride in LD₅₀ is 5 g/kg or more, which shows a high level of safety for the use in human bodies and the like.

No specific limitations are imposed on the dietary fibers used in the present invention, and examples of the dietary fibers include pectin, polydextrose, alginic acid, sodium alginate, galactomannan, glucomannan, tamarind-seed gum, xanthan gum, gelan gum, carrageenan, karaya gum, carboxymethyl cellulose, cellulose, hemicellulose, lignin, insoluble pectin, and composite dietary fibers, such as apple fiber, sugar-beet fiber, corn fiber, wheat fiber, soybean fiber, and chitin. These dietary fibers may be used independently, or in combination of two or more.

Preferable among these are apple fiber, sugar-beet fiber, corn fiber, wheat fiber, and soybean fiber.

The contents of the (A) component and (B) component of the lapactic agent of the present invention can be adjusted according to symptoms. The dose of the lapactic agent of the present invention can be exemplified as 0.1 to 4 g, preferably 0.2 to 2 g of the (A) component, and 1 to 50, preferably 1 to 20 g, of the (B) component.

Although the lapactic agent of the present invention may be administered by weighing and mixing the (A) component and (B) component as they are, it may also be administered in the form of solution, suspension, powder, granules, tablets, or capsules, together with pharmaceutically allowable diluents. Examples of diluents include saccharides such as lactose, sucrose, and glucose; starch; inorganic compounds such as calcium carbonate and calcium sulfate; crystalline cellulose; distilled water; purified water; sesame oil; soybean oil; corn oil; olive oil; and cotton-seed oil; all of which are generally used. In formulating, additives, such as a binder, a lubricant, a dispersing agent, a suspending agent an emulsifying agent, a diluting agent, a buffering agent, an antioxidant, and an antibacterial agent, can be used.

The lapactic agent of the present invention can also be administered by incorporation into food products. Examples of food products that can contain the lapactic agent of the present invention include bread, pasta, noodles, cakes, cookies, biscuits, hamburger steak, meat sauce, jiao-zi, sausages, curry, stew, jam, chocolate, and corn soup. Examples of methods for incorporating the lapactic agent of the present invention in food products include a method of mix it, for example, in the dough in the manufacturing process of the dough for a food product; or, in the case of a liquid food product, a method of dissolving it evenly in the food product. Although the content of the lapactic agent of the present invention in these food products depends on the kind or the ingesting amount of the food products, the content can be, for example, 1 to 20% by weight.

The lapactic agent of the present invention can also be administered by taking a food product wherein the (A) component is mixed into a food product or a food material containing the (B) component, which is a dietary fiber; or by taking a food product containing the (A) component together with a food product or a food material containing the (B) component, which is a dietary fiber.

Formulation examples of the food products containing the lapactic agent of the present invention will be shown below. The unit used in the formulation examples is parts by weight.

Food formulation example 1 (spaghetti dough)

| | | |
|---|---|---|
| Wheat flour | 100 | parts by weight |
| Water | 30 | |
| (A) component | 0.1 to 0.2 | |
| (B) component | 5 to 6 | |

Food formulation example 2 (bread dough)

| | | |
|---|---|---|
| Wheat flour | 100 | parts by weight |
| Water | 64 | |
| Yeast | 3 | |
| Salt | 2 | |
| Sugar | 6 | |
| Powdered skim milk | 3 | |
| Cooking oil | 5 | |
| (A) component | 0.1 to 0.2 | |
| (B) component | 2 to 5 | |

Food formulation example 3 (hamburger steak patty)

| | | |
|---|---|---|
| Ground beef and pork | 40 | parts by weight |
| Beef tallow | 5 | |
| Onion | 20 | |
| Bread crumbs | 8 | |
| Starch | 8 | |
| Water | 5 | |
| Spice, seasoning | 1.2 | |
| (A) component | 0.2 to 0.5 | |
| (B) component | 5 to 15 | |

### EXAMPLES

The present invention will be described below in further detail by reference to examples; however, the present invention is in no way limited by these examples. ε-Polylysine used in these examples is a product of Chisso Corporation.

### Example 1, Comparative Examples 1-3

### Lapactic Effect Test 1

After feeding Solid Feed MW (trade-name of Oriental Yeast Co., Ltd.) for 4 days to male SD rats of 4 weeks old, the rats were divided into 4 groups of 6 animals each. Thereafter, feed of the formulations shown in Table 1 was fed to the rats in each group for 14 days, feces were collected between the 11th day and the 14th day, and the feces were weighed after drying. The results are shown in Table 2. As seen from Table 2, the lapactic agent of the present invention was found to increased the quantity of feces, and to have a lapactic effect.

TABLE 1
(% by weight)

| | Ex. 1 | C. Ex. 1 | C. Ex. 2 | C. Ex. 3 |
|---|---|---|---|---|
| Casein | 15.0 | 15.0 | 15.0 | 15.0 |
| Sugar-beet fiber | 10.0 | | | 10.0 |
| ε-polylysine | 0.4 | | 0.4 | |
| Mineral complex* | 3.5 | 3.5 | 3.5 | 3.5 |
| Vitamin complex* | 1.0 | 1.0 | 1.0 | 1.0 |
| Corn oil | 5.0 | 5.0 | 5.0 | 5.0 |
| Choline chloride | 0.2 | 0.2 | 0.2 | 0.2 |
| Starch | 64.9 | 75.3 | 74.9 | 65.3 |
| Total | 100.0 | 100.0 | 100.0 | 100.0 |

*Compliant with AIN (American Institute of Nutrition)-76 (Reference: American Institute of Nutrition (1977), Report of the American Institute of Nutrition ad Hoc Committee on Standard for Nutritional Studies. J Nutr 107: 1340-8)

TABLE 2

| | Ex. 1 | C. Ex. 1 | C. Ex. 2 | C. Ex. 3 |
|---|---|---|---|---|
| Feces dry weight | 4.690 ± | 1.503 ± | 1.455 ± 0.278 | 3.234 ± 0.441 |
| (g/4 days) | 1.082 | 0.148 | | |

### Example 2

### Lapactic Effect Test 2

The dough of the following formulation was kneaded well, leavened, and baked into bread.

| | | |
|---|---|---|
| Wheat flour | 100 | parts by weight |
| Water | 64 | |
| Yeast | 3 | |
| Salt | 2 | |
| Sugar | 6 | |
| Powdered skim milk | 3 | |
| Cooking oil | 5 | |
| ε-Polylysine | 0.2 | |
| Sugar-beet fiber | 2 | |

Each of twenty males and twenty females in their 20s to 40s who were more or less constipated; i.e., whose stool frequencies were 3 to 5 a week, ingested 180 g of the above-described bread at breakfast and dinner for one week, and the lapactic effect and stool frequencies were observed. The results are shown in Table 3.

As seen from Table 3, administration of the food product containing the lapactic agent of the present invention tends to improve bowel movement.

TABLE 3

| | Evaluation | Number |
|---|---|---|
| Lapactic effect | Much improved | 4 |
| | Improved | 9 |
| | Slightly improved | 5 |
| | No change | 2 |
| | Aggravated | 0 |
| Number of bowel movements | Increased | 16 |
| | No change | 4 |
| | Decreased | 0 |

### EFFECT OF THE INVENTION

The lapactic agent of the present invention has higher lapactic effects such as the promotion of catharsis even in smaller quantity of intake than conventional dietary-fiber-based lapactic agents. Since the lapactic agent of the present invention is effective even in a small quantity of intake, it can be taken comfortably for a long period of time, and the adverse effect of inhibition of systemic absorption of minerals can be minimized. In addition, since food products containing the lapactic agent can lower the content of dietary fibers, the effect of containing dietary fibers, which worsens the feel of food products to the tongue and in the mouth, can be prevented.

1-4. (canceled)

5. A lapactic agent comprising (A) a basic polymer and/or the salt thereof and (B) a dietary fiber that together cause a lapactic effect, wherein (A) a basic polymer and/or the salt thereof is at least one selected from the group consisting of polyvinylamine, polyallylamine, polyethyleneimine, polyvinylpyridine, poly (N,N-dimethylaminoethyl acrylate), a copolymer formed from a monomer having an amino group selected from the group consisting of vinylamine, allylamine, vinylpyridine, vinylmethylimidazole, N,N-dimethylaminoethyl acrylate, and N,N-dimethylaminopropyl acrylate; and a salt of these polymers.

6. The lapactic agent according to claim 5, wherein (B) the dietary fiber is at least one selected from the group consisting of apple fiber, sugar beet fiber, corn fiber, wheat fiber, and soybean fiber.

7. A food product that contains a lapactic agent according to claim 5.

8. A lapactic agent comprising s-polylysine and sugar beet fiber.
